(19) United States
(12) Patent Application Publication
(10) Pub. No.: US 2006/0083748 A1
(43) Pub. Date: Apr. 20, 2006

(54) Calixarenes for use as excipient for an active substance

(76) Inventors: Hans-Uwe Wolf, Neu-Ulm (DE); Jorg Martin Dormann, Blaustein (DE)

Correspondence Address: YOUNG & BASILE, P.C., 3001 WEST BIG BEAVER ROAD, SUITE 624, TROY, MI, 48084, US

(21) Appl. No.: 10/518,111

(22) PCT Filed: Jun. 4, 2003
(86) PCT No.: PCT/EP03/05830

§ 371 Date: Sep. 16, 2005

(30) Foreign Application Priority Data
Jun. 12, 2002 (DE) 102 26 098.2

(51) Int. Cl. A61K 39/00; C07H 21/00; A61K 47/00

(52) U.S. Cl. 424/184.1; 514/772; 534/653; 536/22.1; 536/46

(57) ABSTRACT

The invention relates to an active substance excipient system, wherein the excipients are selected from the group of calixarenes or resorcinarenes. Said macromolecular excipients serve for transport of the active substance to the location of action and to a dosage and time-defined release of the same there. Metabolism, kinetics and release mechanism can be controlled by chemically modifying the excipient in a purposeful manner.

The invention relates to an excipient system for an active substance, in which excipients are selected from the group of calixarenes or resorcinarenes. These macromolecular excipients serve firstly to transport this active substance to the target and secondly to release it there with respect to dosage and time. Through selective chemical modifications to the excipient, metabolism, kinetics and release can be controlled.

The topical use of active substances and their optimization has played a great role in pharmceutical research for years. Pharmacokinetic aspects, such as transport, distribution and release of the active substance are in the foreground. Solutions known from the prior art have been based so far on deriving the active substance in accordance with pharmacokinetic prerequisites. A further approach was based on optimizing the galenics of the medication for the particular application.

In the same way, excipient systems have been used previously which enabled the transport of the active substance to the desired target. A number of important aspects must be taken into consideration here:

1. Resorption:

Substance properties (such as, for example, water solubility, lipophilia, molecular size, acid or base character, galenics and interactions with other substances (synergistic or antagonistic) play a major role here.

2. Distribution:

Factors such as pharmacokinetics and membrane permeation (e.g. through diffusion, filtration, carrier-mediated transport or vascular transport) are foremost.

3. Retention: (Bonding)

4. Elimination:

This concerns primarily the metabolic breakdown of the substances.

It has not yet been possible to achieve a satisfactory solution to all these aspects for an excipient system.

With this as the starting point, the objective of the present invention was to provide an excipient system for an active substance which eliminates the disadvantages of the prior art and with which both the transport and the release of the active substance can be controlled in a selective manner.

This objective is achieved with the excipient system for an active substance with the features of claim 1. The additional dependent claims reveal advantageous refinements. The use of calixarenes or resorcinarenes as excipient systems for active substances is described in claim 9.

In accordance with the invention, an active substance excipient is provided which [consists of] of at least one carrier molecule from the group of calixerenes with the general formula I 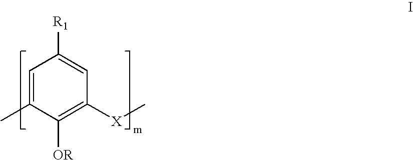
with R═H, alkyl, aryl, alkyloxy, aryloxy, amin, amide, carbonic acids and sulphonic acids with 1 to 12 C-atoms, amino acids, glucose or crown ethers,
R₁═H, alkyl, aryl, alkyloxy, aryloxy, amin, amide, carbonic acids and sulphonic acids with 1 to 12 C-atoms, sulphonic amides, amino acids, glucose, crown ethers, cyclodextrins, purine bases, pyrimidine bases or azophenyl dyes,
X=methylene, S, O, N, P or Si and m=4, 5, 6 or 8, wherein the aromatic systems may have heteroatoms, for example, as a pyridine derivative,

and/or the resorcinarenes with the general formula II 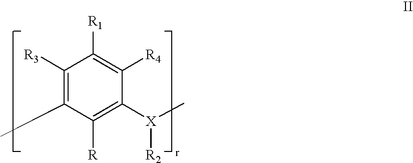
with R═H, alkyl, aryl, alkyloxy, aryloxy, amin, amide, carbonic acids and sulphonic acids with 1 to 12 C-atoms or amino acids,
R₁═H, alkyl, aryl, alkyloxy, aryloxy, amin, amide, carbonic acids and sulphonic acids with 1 to 12 C-atoms, sulphonic amides, amino acids, glucose, crown ethers, cyclodextrins, purine bases, pyrimidine bases or azophenyl dyes,
R₂=alkyl or aryl,
X=methylene, S, O, N, P or Si,
r=4, 5, 6 or 8,
and R₃=hydroxyl and R₄═H
or
R₃ and R₄=0, where R₃ and R₄ are bridged via methylene, ethylene or quinoxaline,
where the aromatic systems may have heteroatoms, for example, as pyridine or an oxazole derivate,

and at least one active substance.

The following compounds can be used as azophenyl dyes:

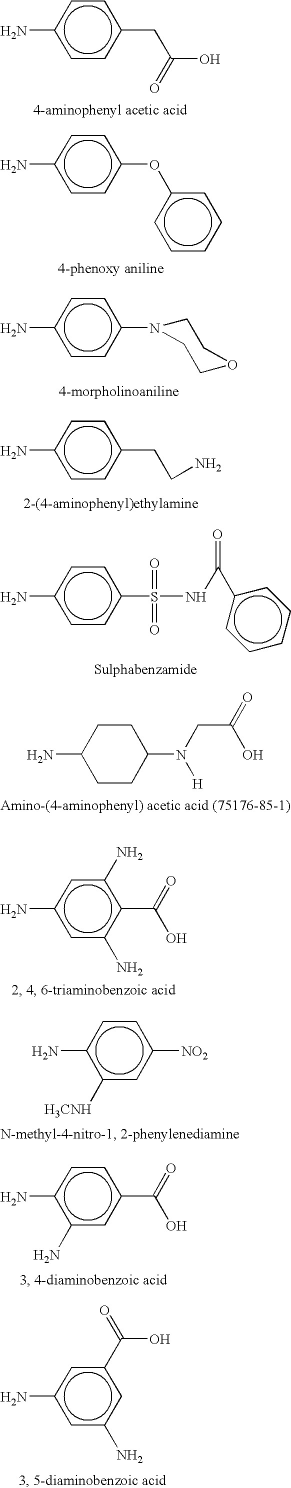

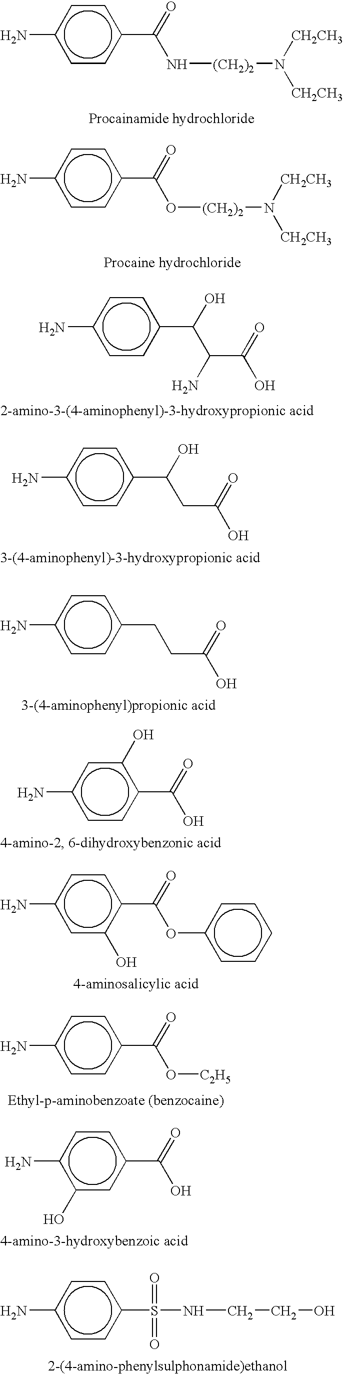

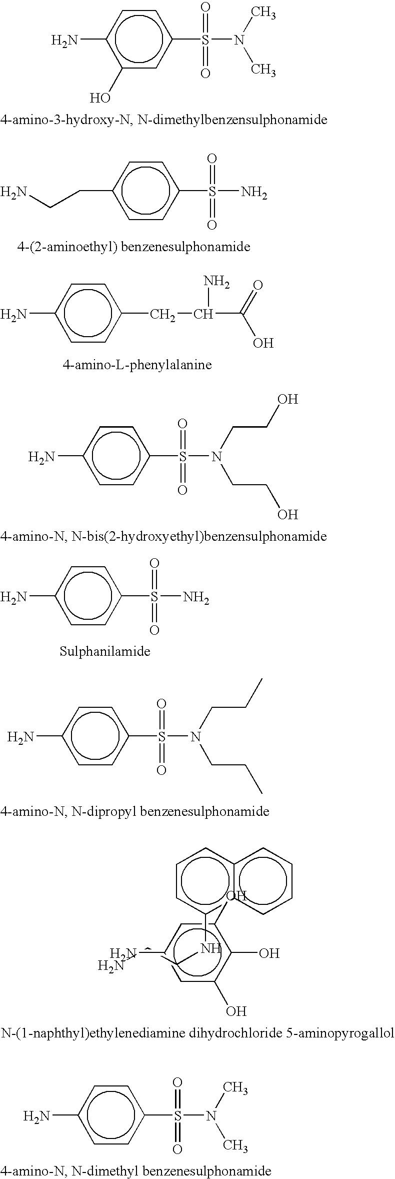

It is a unique feature of the solution in accordance with the invention that it is not the active substance but the synthetic excipient system that is modified such that the active substance can be transported to each desired target and released there with respect to dosage and time.

The water solubility of the active substance can be increased by the introduction of functional groups, such as, for example, sulphonic acid, carbonic acid, alcohol and amin groups.

The excipient system is preferably modified in such a way that it represents a second-order metabolite. Sulphonic acid or glucoronic acid groups are suitable for achieving this modification. In this case, the excipient system is present as a second-order metabolite, so that renal elimination of the same from the body is possible. Very short residence times for the excipient in the body can be achieved thereby. On the other hand, if lipophilic excipients are used, they have a considerably longer residence time.

The excipient system can similarly be modified in such a way that selective release of the active substance from the excipient system is possible. The release can thereby be time-controlled in such a way that the entire time scale from an immediate release of the entire quantity of the active substance up to a sustained continuous release is possible. The possible ways of modifying control of the release are:

1. Physical chemically active control of the method and strength of the interaction between active substance and carrier,

2. Physical chemically induced control by neutralizing interactions between active substance and carrier, for example, by changing the pH,

3. Passive control by neutralizing interaction between individual modules in multi-component carriers, known as capsules and cages, and

4. Metabolic control over the partially metabolic breakdown of the excipient.

A particular advantage of the excipient is based on the fact that it can be degraded enzymatically, for example, by aldolases, ketolases, esterases and cytochrome P450 and at the same time the active substance can be released.

Three different types are available for enzymatic breakdown.

With type A, starting from compounds with the general formula I with R=alkyl, aryl, alkoxy or aryloxy and X=methylene, there is a cleavage of the bond between the aromatic system and the OR remainder. As a result of this cleavage, the frozen cone formation is dissolved, resulting in the release of the active substance. This is also known as a flip-off mechanism. The bonds are attacked in particular by cytochrome P 450, breakdown is not stearically inhibited and takes place rapidly.

With type B, starting from compounds with the general formula I with X═S, P, N, Si, there is enzymatic cleavage of the thiole bond, resulting in ring opening of the calixarene or resorcinarene which enables the release of the active substance.

Type C refers to only resorcinarenes. With bridged compounds of this type, one also speaks of “cavitands.” Because of the cup-shaped structure of the resorcinarenes, compared with the calix-shaped calixerenes, the cup-shaped structure in the former is converted through an additional bridge bond resulting by way of the unit X, into a calix-shaped structure which enables the inclusion of the active substance. By enzymatic cleavage at X, this bridge structure can be dissolved, whereby the resorcinarene passes back again into the cup-shaped structure which enables the release of the active substance.

A further advantageous embodiment provides for the excipient to be modified by means of an enzymatically degradable linker and it thus is present as a prodrug.

As another alternative, the carrier can be modified with receptor-analogous groups which can be broken down statically by endocytosis. Here in particular, amino acids and glucose modifications should be mentioned. Optimization to the particular receptor is basically possible.

Preferably the active substance is covalently bonded to the excipient. But in the same way, it is just as possible that the active substance is bonded by way of a spacer to the excipient. Peptide and nucleotide spacers in particular, which are selectively degradable enzymatically, act as spacers.

It must be considered a particular advantage of the excipient system for the active substance that manufacture uses a limited number of a few central active substances which have minor or no side effects. This results firstly in a cost reduction, secondly a reduction in the number of active substances is advantageous from a medical perspective regarding the avoidance or suppression of side effects. There is an additional advantage that the excipient can be selectively optimized with respect to the particular target, since the functionalization of the present compounds is easily possible. In the same way, pharmacokinetics and metabolic breakdown can easily be controlled.

Based on the following examples, the object in accordance with the invention will be explained in more detail without limiting it to the embodiments named.

### EXAMPLE 1

Production of p-tert-butylcalix[4]arenas

Production is carried out according to the reaction schematic shown in formula III:

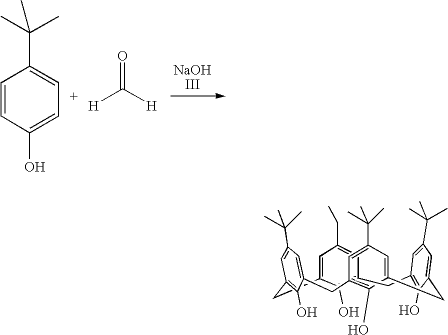

The following compounds are used for the synthesis:

| | | | |
|---|---|---|---|
| p-tert butylphenol | (1.332 mol) | 200 | g |
| Formaline solution (37% in H₂O) | (1.66 mol) | 125 | ml |
| Sodium hydroxide | (60 mmol) | 2.4 | g |
| Water | | 8 | ml |
| Diphenylether | | 2 | l |
| Ethylacetate | | 3 | l |
| Acetic acid, water and acetone for washing. | | | |

In a 4-liter 3-necked flask with a KPG® stirrer, water separator, return cooler and a gas inlet and exhaust device, the mixture of p-tert-butylphenol, formaline solution and aqueous sodium hydroxide solution is stirred vigorously for 20 minutes at room temperature until the white mixture has a homogenously papescent consistency. Then it is heated to 120° C. with the aid of a heating bath. While it is being heated, a stream of nitrogen is blown through the apparatus to accelerate the separation of the water. After a short time the reaction mixture can be observed to have a slight yellow coloration. After about 1 hour, the mixture, which is becoming increasingly viscous, foams so that half the flask is filled. After another hour of heating at 120° C. under a weak nitrogen stream, the contents of the flask, now beige, solidifies like glass. It is allowed to cool to room temperature and the contents of the flask are absorbed in diphenylether and stirred again at 80° C. until the residue is completely dissolved. The temperature is increased to 160° C. and a strong stream of nitrogen is blown through the apparatus to expel the water completely. The color of the reaction mixture changes from beige to black. When hardly any more water is being separated, the heating bath is replaced by a heating mantle and the water separator by an intensive cooler, and the reaction mixture is heated for 4 hours with reflow (260° C.) and under a weak nitrogen stream. Then the contents of the flask are cooled to room temperature, mixed with 2.5 l of ethylacetate and stirred overnight. A brown sediment precipitates out which is vacuumed off and washed with ethylacetate. The light-brown raw product is washed successively with 500 ml of acetic acid (30%), twice with 500 ml of water and once with 100 ml of acetone. The residue is heated in 1 liter of toluene for 3 hours with return flow, vacuumed off, washed with acetone and dried in a vacuum oil pump. The product is white and finely granulated.

The yield was 71.8 g (68% per liter)

The following analytical characteristics could be determined:

IR (KBr, RT):
3130 (ν_{OH});3052, 3023 (ν_{Aryl-H}); 2961, 2905 2869 (ν_{CH2}); 1605 (ν_{C═C}); 1481 (δ_{CH2}).
¹H-NMR (400 MHz, CDCl₃ with TMS): 10.33 (s, 4H, Ar—OH); 7.05 (s, SH, Ar—H); 4.26 and 3.49 (d, 8H, Ar—CH2-Ar); 1.21 (s, 36H, CH₃)
¹³C-NMR (400 MHz), CDCl₃ with TMS): 146.67 (C—OH); 144.38 (C-t-butyl); 127.69 (Cₐᵣ—CH₂—); 125.93 (Cₐᵣ—H); 60.39 (C—CH₃); 34.00 (Ar—CH₂—Ar); 31.40 (CH₃).
MS (negative FAB):
   m/e=647 [M−H]⁻ (calculated for C₄₄H₅₆O₄: M=648.9 g/mol)

### EXAMPLE 2

Production of calix[4]arenes

Production is carried out according to the reaction schematic shown in formula IV:

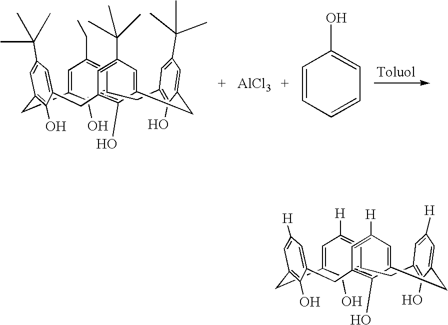

The following compounds were used to start:

| | | | |
|---|---|---|---|
| p-tert butylcalix[4]erene | (100 mol) | 65 | g |
| AlCl₃ (water-free) | (530 mol) | 71 | g |
| Phenol (dry) | (466 mol) | 44 | g |
| Toluene (absolute) | | 900 | ml |
| 1 N hydrochloric acid | | 1.8 | l |
| Sodium sulphate for drying | | | |

A 2-liter 3-neck flask equipped with a gas inlet and exhaust device and a drying tube is annealed, evacuated several times and purged with argon. Then calix[4]arenes are mixed with phenol in a protective gas atmosphere. With humidity excluded and while being stirred vigorously, toluene and aluminum trichloride are added, whereby the mixture changes into a brown-orange clear solution. The contents of the flask are stirred for 4 hours; it becomes increasingly cloudy and a beige sediment forms. The reaction is stopped by the addition of hydrochloric acid; the two resulting beige phases are stirred overnight. The phases, now clear, are separated and the organic phase is thoroughly agitated once with water. After drying the organic phase with sodium sulphate and adding methanol, the raw product precipitates out in the form of a white, crystalline solid. This is vacuumed off and crystallized into methylene chloride/methanol. Drying in the vacuum oil pump delivers the white, finely granulated product.

The yield was 37.52 g (88.5% per liter)

The following analytical characteristics could be established:

IR (KBr, RT):
3150 (ν_{OH}); 3092, 3054 (ν_{Aryl-H});2931, 2866 (ν_{CH2}); 1608 (ν_{C═C}); 1466 (δ_{CH2}).
¹H-NMR (400 MHz, CDCl₃ with TMS δ=0 ppm): 7.04 (d, 8H, Ar—H); 6.72 (t, 4H, Ar—H); 4.26 and 3.54 (br s, 8H, Ar—CH₂—Ar);
¹³C-NMR (400 MHz), CDCl₃ δ=77 ppm with TMS): 148.77 (C—OH); 128.96 (Cₐᵣ—CH₂—); 128.23 (Cₐᵣ); 122.22 (Cₐᵣ); 31.69 (Ar—CH₂).
MS (negative FAB):
   m/e=423 [M−H]⁻ (calculated for C₂₈H₂₄O₄: M=424.5 g/mol)

After the hydrochloric acid is added to stop the reaction, it is absolutely necessary to stir the reaction mixture vigorously for more than 6 hours. If this is not done, a slightly contaminated, yellow-greenish product is obtained. The contamination is an aluminum-organic compound which is not characterized more closely.

### EXAMPLE 3

Production of 5,11,17,23-tetrasulphonic acid calix[4]arenes

Production is carried out according to the reaction schematic shown in formula V:

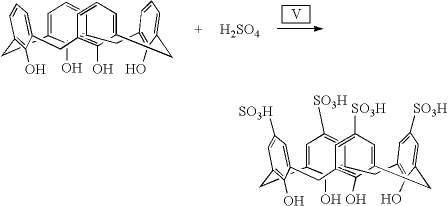

The following compounds were used to start:

| | | |
|---|---|---|
| Calix[4]arenes | (7 mmol) | 3 g |
| Sulfuric acid (98%) | | 30 ml |
| Methanol, ethylacetate | | |

In a 100-ml 3-necked flask with a gas inlet and exhaust device, the sulfuric acid is added all at once to the the calix[4]arenes. The apparatus is purged with argon and the reaction mixture is stirred at 80° C. for about 4 hours. The progress of the reaction is followed by taking samples and solubililty testing in water. When the mixture is soluble in water with no residue, the reaction is stopped. The raw product is vacuumed off with a glass frit (4A), dissolved in methanol (to remove any remaining sulfuric acid) and precipitated with ethyl acetate. The white sediment is dried in a vacuum oil pump.

The yield was 4.2 g (68% per liter).

The following analytical characteristics could be established:

IR (KBr, RT):
3372, 3224, 3125 (ν_{OH}); 1473, 1461 (δ_{CH2}), 1171 (ν_{SO2}).
¹H-NMR (400 MHz, D₂O, δ=4.65 ppm): 7.40 (S, 8H, Ar—H); 3.82 (br s, 8H, Ar—CH₂—Ar); ¹³C-NMR (400 MHz), CDCl₃ δ=50.2 ppm): 153.29 (C—OH); 137.24 (C—SO₃H); 129.83 (Cₐᵣ—CH₂—); 128.03 (Cₐᵣ); 32.05 (Cₐᵣ—CH₂—Cₐᵣ).
MS (MALDI-TOF):
   m/e=767.1 [M+nA]⁺ (calculated for C₂₈H₂₄O₁₆S₄: M=744.8 g/mol)

### EXAMPLE 4

The absorption of orally administered active substances can be determined by their ability to pass the gastrointestinal tract. Depending on their molecular properties, active ingredients can choose either the transcellular or the paracellular path or, in a few exceptions, also follow an active transport mechanism. In the case of the latter variants, which are usually not accessible in an artificial membrane system, a new test system was developed as part of this study (the PAMPORE system), which has hydrophilic pores in the lipid layer. The PAMPORE system was developed and patented by the Pharmacelsus CRO Company in Saarbrücken. The related studies were conducted by Pharmacelsus CRO.

Components which select the transcellular path were studied using a traditional artificial membrane without hydrophilic pores. The combination of these two test systems allows the study of the permeability of a plurality of active ingredients independently of the type of transport which they prefer.

All active ingredient excipient systems were first produced as a 2.5 or 5 mM standard solution in ethanol or Tris-buffer. In a next step, diluted in the Tris-buffer as if to a final concentration of 125 or 250 μM at a pH of 7.4. The time of permeation through the artificial membrane was 16 hours. In Table I below, individual calixarenes are listed as active substance excipients with their membrane permeation capability.

TABLE 1

| | Membrane permeation | |
|---|---|---|
| | (%) | |
| | Standard | PAMPORE |
| Tetra [dimethylamino-methyl]-calix[4]arenes | <10 | 94 |
| Tetrasulphonic acid calix[4]arenes | <15 | 100 |
| Hexasulphonic acid calix[6]arenes | 0 | 100 |
| Tetramethyltetramethyl resorcin[4]arenes | <15 | 95 |
| p-tert-butyltetra acetic acid calix[4]arenes | 16 | 88 |

Using the PAMPORE system, after hydrophilic pores are present in the lipid layer, all five excipient systems have migrated almost completely through the membrane by the paracellular path. On the other hand, the intact lipid membranes without hydrophilic pores can be passed by the excipient systems only to a very minor degree by the transcellular path.

### EXAMPLE 5

Model substance for the passive release (without enzymes, Type 1): calix[4]arene-tetrasulphonic acid complex with aciclovir (Formula VI)

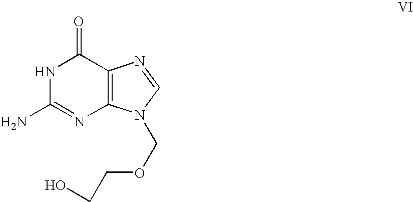

Activity:

Herpes simplex 1>herpes simplex 2>varicella zoster; against Epstein-Barr only in vitro, not active against CMV in achievable concentrations.

Pharmacokinetics:

Administration i.v. or oral, absorption in the intestinal tract only 15-20%, with great variability. For optimal effect, administration necessary 5 times daily. Elimination: primarily through kidneys, extended t/2 with renal insufficiency (from 3 hours with normal function to 18 hours with anuria), adjustment of the dosage interval (from 8 hourly to 12 hourly to 24 hourly). Good distribution in the entire body, liquor about 20-50% of the serum concentrations.

Application:

Mucocutaneous herpes simplex infections: accelerated healing of lesions, virus elimination, symptoms; overall moderate benefit

Recurring mucocutaneous herpes infections: Chron. suppressive therapy reduces rate of attack

Herpes simplex keratitis (topical and systemic)

Herpes simplex encephalitis: high dosage

Neonatal herpes infection

Varizella zoster infections: faster healing (symptoms, lesions, pain), no clear effect on post-herpetic neuralgia (effect overall marginal)

With immune suppressed patients (AIDS, chemotherapy); prevents dissemination, faster healing; i.v. administration

This complex was chosen because the bio-availability of aciclovir is relatively poor and should be markedly improved by the carrier. A delayed effect, or ‘slow drug release’, is to be effected.

A 1:1 complex is formed. The complex formation is in the range 10³.

1. Excipient system for an active substance consisting of at least one carrier molecule from the group of calixarenes with the general formula I
I 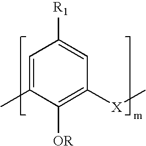
with R═H, alkyl, aryl, alkyloxy, aryloxy, amin, amide, carbonic acids and sulphonic acids with 1 to 12 C-atoms, amino acids, glucose or crown ethers,
R1=H, alkyl, aryl, alkyloxy, aryloxy, amin, amide, carbonic acids and sulphonic acids with 1 to 12 C-atoms, sulfonamides, amino acids, glucose or crown ethers, cyclodextrin, purine bases, pyramidine bases or azophenyl dyes,
X=methylene, S, O, N, P or Si and
m=4, 5, 6 or 8,
wherein the aromatic systems may have at least one of heteroatoms and resorcinarenes with the general formula II
II 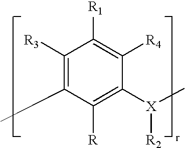
with R═H, alkyl, aryl, alkyloxy, aryloxy, amin, amide, carbonic acids and sulphonic acids with 1 to 12 C-atoms or amino acids,
R1=H, alkyl, aryl, alkyoxyl, aryloxy, amin, amide, carbonic acids and sulphonic acids with 1 to 12 C-atoms, sulphonamides, amino acids, glucose or crown ether, cyclodextrin, purine bases, pyramidine bases or azophenyl dyes,
R₂=alkyl or aryl,
X=methylene, S, O, N, P or Si and
r=4, 5, 6 or 8,
and
R₃=hydroxyl and R₄═H
or
R₃ and R₄=0, where R₃ and R₄ are bridged by way of methyls, ethyls or quinoxaline,
wherein the aromatic systems may have heteroatoms, and at least one active substance.

2. Excipient system for an active substance according to claim 1, wherein the carrier is modified to increase water solubility, in particular by at least one of sulphonic acid groups, carbonic acid groups, amino groups and alcohols.

3. Excipient system for an active substance according to claim 1, wherein the carrier is modified to influence the pharmacokinetics of the system, in particular by one of sulphonic acid groups, and glucuronic acid groups and is a second-order metabolite.

4. Excipient system for an active substance according to claim 1, wherein the carrier is enzymatically degradable while releasing the active substance, in particular by aldolases, ketolases, esterases and cytochrome P 450.

5. Excipient system for an active substance according to claim 1, wherein the carrier is modified by means of a linker which can be broken down enzymatically and is present as a prodrug.

6. Excipient system for an active substance according to claim 1, wherein the carrier is modified by means of receptor-analogous groups which can be broken down statically by endocytocis.

7. Excipient system for an active substance according to claim 1, wherein the active substance is covalently bonded to the carrier.

8. Excipient system for an active substance according to claim 1, wherein the active substance is bonded to the carrier through a spacer, for example, one of a nucleotide spacer and a peptide spacer.

9. Use of at least one of calixerenes and resorcinarenes with the general formula I or II in claim 1 as excipient systems for active substances.
